(19) United States
(12) Patent Application Publication
(10) Pub. No.: US 2024/0352459 A1
(43) Pub. Date: Oct. 24, 2024

(54) COMPOSITIONS AND METHODS FOR SIRNA TREATMENT OF MUSCULAR DYSTROPHY

(71) Applicant: UNIVERSITY OF MASSACHUSETTS, Boston, MA (US)

(72) Inventors: Charles P. EMERSON, Lyndon, VT (US); Katelyn DAMAN, Stow, MA (US); Jing YAN, Shrewsbury, MA (US); Anastasia KHVOROVA, North Worcester, MA (US); Annabelle BISCANS, North Worcester, MA (US); Julia ALTERMAN, North Worcester, MA (US)

(21) Appl. No.: 18/682,646

(22) PCT Filed: Aug. 9, 2022
(86) PCT No.: PCT/US2022/039818

§ 371 Date: Feb. 9, 2024

Related U.S. Application Data
(60) Provisional application No. 63/231,328, filed on Aug. 10, 2021.
(60) Provisional application No. 63/249,700, filed on Sep. 29, 2021.

(51) Int. Cl. C12N 15/113; A61K 31/713; A61K 47/54

(52) U.S. Cl. CPC C12N 15/113; A61K 31/713; A61K 47/543; C12N 2310/14; C12N 2310/3125; C12N 2310/321; C12N 2310/322

(57) ABSTRACT

The present invention is related to the treatment of muscular dystrophy. In particular, compositions of short interfering ribonucleic acids (siRNAs) Eire contemplated that are targeted to different portions of a DUX4 messenger ribonucleic acid molecule. Preferably, these siRNAs are double stranded having a sense strand of fifteen (15) nucleotides. When applied as a therapeutic method, the short interfering ribonucleic acids reduce DUX4 protein translation and mediate a therapeutic response in human facioscapulohumeral muscular dystrophies, FSHD 1 and FSHD2.

### CROSS-REFERENCE TO RELATED APPLICATIONS

The present application claims the benefit of U.S. Provisional Patent Application Nos. 63/231,328, filed on Aug. 10, 2021 and 63/249,700, filed on Sep. 29, 2021, which are both incorporated herein by reference.

### STATEMENT OF GOVERNMENTAL INTEREST

This invention was made with government support under Grant No. HD060848 awarded by The National Institutes of Health, Institute of Child Health And Human Development. The government has certain rights in the invention.

### FIELD OF THE INVENTION

The present invention is related to the treatment of muscular dystrophy. In particular, compositions of short interfering ribonucleic acids are contemplated that are targeted to different regions of a DUX4 messenger riboucleic acid (mRNA) molecule. When applied as a therapeutic method, the short interfering ribonucleic acids reduce DUX4 protein translation by inducing DUX4 mRNA degradation to mediate a therapeutic response in human facioscapulohumeral muscular dystrophy.

### BACKGROUND

Muscular Dystrophy (MD) incorporates an assortment of hereditary disorders that lead to progressive, generalized disease of the muscle prompted by mutations that disrupt muscle contractile protein function or muscle membrane integrity or cause toxicities that cause muscle destruction. Muscular dystrophy is a non-communicable disorder with abundant variations. Each has its pattern of inheritance, onset period, and the rate at which muscle is lost. Alterations in specific genes cause different representations of this disease.

Onset is variable for different diseases and individuals with a genetic disease, which may be revealed in infancy or undergo accelerated deterioration near the age of onset. Parents of affected individuals may present concern that their child is not walking as well as other children their age. The child may have trouble kicking a ball, smiling, sitting, raising arms above shoulders, and breathing. Other complaints can involve a history of delayed ambulation, toe walking, calf hypertrophy, and proximal hip girdle muscle instability. Currently available treatments include pharmaceuticals, surgery or musculoskeletal bracing. LaPelusa et al., “Muscular Dystrophy”; In: StatPearls (Intenet) Treasure Island (FL): StatPearls Publishing; May 26, 2021; ncbi.nlm.nih.gov/books/NBK560582/#article-25401.s9.

Presently, none of these treatment options has been successful in reducing or eliminating either the symptoms or addressing the genetic causation of these conditions. There is currently no FDA approved treatment or cure for MD with standard of care treatments aiming to alleviate symptoms. Therefore, therapeutic development for MD is a critical unmet need.

### SUMMARY

The present invention is related to the treatment of facioscapulohumeral muscular dystrophies, FSHD1 and FSHD2. In particular, compositions of short interfering ribonucleic acids are contemplated that are targeted to different regions of a DUX4 mRNA molecule to destroy the ability to translate the toxic DUX4 protein. When applied as a therapeutic method, the short interfering ribonucleic acids reduce DUX4 protein translation to mediate a therapeutic response in patients with human facioscapulohumeral muscular dystrophies FSHD1 and FSHD2.

In one embodiment, the present invention contemplates a composition comprising a double stranded DUX4-mRNA targeted short interfering ribonucleic acid (siRNA) comprising a sense strand consisting of fifteen (15) nucleotides. In one embodiment, the double stranded DUX4-mRNA targeted siRNA further comprises an antisense strand consisting of twenty (20) nucleotides. In one embodiment, the sense strand is complementary to at least a portion of a DUX4 mRNA target sequence. In one embodiment, the double stranded DUX4-mRNA targeted siRNA is conjugated to a lipid. In one embodiment, the lipid is docosanoic acid or cholesterol. In one embodiment, the composition further comprises a pharmaceutically acceptable composition. In one embodiment, said sense strand consists of the sequence UUACAUCUCCUGGAU (SEQ ID NO: 1). In one embodiment, said antisense strand consists of the sequence UUUAAUAUAUCUCUGAACUA (SEQ ID NO: 2). In one embodiment, said sense strand consists of the sequence GGAUUAGAGUUACAU (SEQ ID NO:3). In one embodiment, said antisense strand consists of the sequence UCUCUGAACUAAUCAUCCAG (SEQ ID NO: 4). In one embodiment, said sense strand consists of the sequence AGAGUUACAUCUCCU (SEQ ID NO: 5). In one embodiment, said antisense strand consists of the sequence AUAUAUCUCUGAACUAAUCA (SEQ ID NO:6). In one embodiment, said sense strand consists of the sequence CUGGAUUAGAGUUAC (SEQ ID NO:7). In one embodiment, said antisense strand consists of the sequence UCUGAACUAAUCAUCCAGGA (SEQ ID NO: 8). In one embodiment, said SEQ ID NO: 1 is complementary to at least a portion of a UAGUUCAGAGAUAUAUUAAA (SEQ ID NO:9) target sequence or at least of portion of a AUGAUUAGUUCAGAGAUAUAUUAAAAUGCC (SEQ ID NO: 10) target sequence. In one embodiment, said SEQ ID NO: 3 is complementary to at least a portion of a CUGGAUGAUUAGUUCAGAGA (SEQ ID NO: 11) target sequence or at least a portion of a AUCUCCUGGAUGAUUAGUUCAGAGAUAUAU (SEQ ID NO:12) target sequence. In one embodiment, said SEQ ID NO: 5 is complementary to at least a portion of a UGAUUAGUUCAGAGAUAUAU (SEQ ID NO: 13) target sequence or at least a portion of a CUGGAUGAUUAGUUCAGAGAUAUAUUAAAA (SEQ ID NO: 14) target sequence. In one embodiment, said SEQ ID NO: 7 is complementary to at least a portion of a UCCUGGAUGAUUAGUUCAGA (SEQ ID NO: 15) target sequence or at least a portion of a ACAUCUCCUGGAUGAUUAGUUCAGAGAUAU (SEQ ID NO:16) target sequence.

In one embodiment, the present invention contemplates a method, comprising: a) providing; i) a patient exhibiting at least one symptom of a muscular dystrophy disease and comprising a DUX4 messenger ribonucleic acid (mRNA) target sequence; and ii) a pharmaceutically acceptable composition comprising a double stranded DUX4-mRNA targeted short interfering ribonucleic acid (siRNA) comprising a sense strand that consists of fifteen (15) nucleotides; and b) administering the pharmaceutically acceptable composition to the patient such that the at least one symptom of the muscular dystrophy disease is reduced. In one embodiment, the method further comprises hybridizing the double stranded DUX4-mRNA targeted siRNA to at least a portion of the DUX4 mRNA target sequence. In one embodiment, the muscular dystrophy disease is facioscapulohumeral muscular dystrophy. In one embodiment, the double stranded mRNA siRNA is conjugated to a lipid. In one embodiment, the lipid is docosanoic acid or cholesterol. In one embodiment, the sense strand consists of SEQ ID NO: 1. In one embodiment, the sense strand consists of SEQ ID NO: 3. In one embodiment, the sense strand consists of SEQ ID NO: 5. In one embodiment, the sense strand consists of SEQ ID NO: 7. In one embodiment, said DUX4 mRNA target sequence is or consists of UAGUUCAGAGAUAUAUUAAA (SEQ ID NO:9). In one embodiment, said DUX4 mRNA target sequence is or consists of AUGAUUAGUUCAGAGAUAUAUUAAAAUGCC (SEQ ID NO: 10). In one embodiment, said DUX4 mRNA target sequence is or consists of CUGGAUGAUUAGUUCAGAGA (SEQ ID NO: 11). In one embodiment, said DUX4 mRNA target sequence is or consists of AUCUCCUGGAUGAUUAGUUCAGAGAUAUAU (SEQ ID NO:12). In one embodiment, said DUX4 mRNA target sequence is or consists of UGAUUAGUUCAGAGAUAUAU (SEQ ID NO: 13). In one embodiment, said DUX4 mRNA target sequence is or consists of CUGGAUGAUUAGUUCAGAGAUAUAUUAAAA (SEQ ID NO: 14). In one embodiment, said DUX4 mRNA target sequence is or consists of UCCUGGAUGAUUAGUUCAGA (SEQ ID NO: 15). In one embodiment, said DUX4 mRNA target sequence is or consists of ACAUCUCCUGGAUGAUUAGUUCAGAGAUAU (SEQ ID NO:16).

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “muscular dystrophy” as used herein, refers to any medical condition diagnosed by the results of muscle biopsy, increased creatine phosphokinase (CKM), electromyography, genetic testing, physical examination and medical history. Other diagnostic tests include, but are not limited to, chest X-ray, echocardiogram, computed tomography and magnetic resonance imaging.

The term “muscle cell” as used herein, refers to any type of cell associated with the heart muscle, or skeletal muscle for these tissues. Examples include muscle fibers, myotubes, myocytes, myoblasts, satellite cells and cardiomyocytes.

The term “genetic mutation” as used herein, refers to a permanent alteration in the DNA sequence that makes up a gene, such that the sequence differs from a wild type gene. Mutations range in size; they can affect anywhere from a single base pair to a large segment of a chromosome (e.g., repeat expansions and deletions).

The term “pathogenic variation” as used herein, refers to a genetic alteration that increases an individual's susceptibility or predisposition to a certain disease or disorder. When such a variant (or mutation) is inherited, development of symptoms is more likely, but not certain. Pathogenic variations range in size; they can affect anywhere from a single base pair to a large segment of a chromosome (e.g., 4qA allele in the DUX4 locus on Chromosome 4; (PMID 20724583)).

The term “DUX4” as used herein, refers to the double homeobox 4 gene and is a 424 amino acid double homeodomain-containing pioneer transcription factor encoded by a D4Z4 unit. DUX4 is involved in zygotic genome activation at the cleavage stage. Deregulated expression of DUX4 in muscle and other somatic cells likely underlies FSHD1 and FSHD2 disease.

The term “D4Z4” as used herein, refers to a subtelomeric macrosatellite array of D4Z4 units at 4q35 and 10q26. Normally epigenetically repressed, truncation of the array and/or mutations in genes responsible for maintaining epigenetic repression, allows transcription of DUX4 from a retrotransposed open reading frame in the distal-most D4Z4 unit.

The term “DUX4-s” as used herein, refers to a truncated 159 amino acid splice variant of DUX4 that is identical to DUX4 at the N-terminus so contains the two homeodomains, but is shortened (hence DUX4-s) and lacks the C-terminal transactivation domain of DUX4.

The term “DUX4c” as used herein, refers to a transcription factor largely similar to DUX4 but with a unique C-terminus. Encoded by an inverted and truncated D4Z4 unit located approximately 42 kb centromeric of the D4Z4 array at 4q35.

The term “DUX4 target gene signature” as used herein, refers to a biomarker of direct and indirect genes that are significantly activated by DUX4, usually derived by differential expression analysis in DUX4 over-expression studies in human myogenic cells in vitro. Increased mean expression of all genes in a DUX4 target gene signature indicates that DUX4 is active, or was recently active, in the sample.

The term “FSHD1” as used herein, refers to an inherited muscular dystrophy characterized by a descending skeletal muscle weakness and wasting that often displays left/right asymmetry. Caused by loss of D4Z4 units at 4q35 to between 1 and 10 on at least one allele leading to epigenetic derepression that permits aberrant transcription of DUX4 from the distal-most D4Z4 unit in skeletal muscle. DUX4 transcripts are then spliced and polyadenylated at a downstream poly(A) signal in the pLam region of permissive 4qA haplotypes to stabilize the DUX4 mRNA transcript, leading to production of DUX4 protein, resulting in muscle toxicity.

The term “FSHD2” as used herein, refers to a rare digenic variant of FSHD comprising ˜5% of FSHD cases. Mainly caused by mutations in SMCHD1 that leads to epigenetic derepression at 4q35 when ˜12-16 D4Z4 units are present on at least one allele. This permits transcription of DUX4 from the distal-most D4Z4 unit that is then stabilized and translated due to splicing to a poly(A) signal on permissive 4qA haplotypes.

The term “FSHD target genes” as used herein, refers to 237 DUX4 regulated genes expressed in muscle biopsies of FSHD patients as well as myotubes derived from muscle biopsies of FSHD1 and FSHD2 patients as well as from immortalized FSHD B-lymphoblastoid cell lines. Increased mean expression of all genes distinguishes FSHD from control muscle biopsies.

The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀a with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “associated with” or “linked to” as used herein, refers to an art-accepted causal relationship between a genetic mutation and a medical condition or disease. For example, it is art-accepted that a patient having an HTT gene comprising a tandem CAG repeat expansion mutation has, or is a risk for, Huntington's disease.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “portion” or “at least a portion” when used in reference to a nucleotide sequence refers to a subset of that nucleotide sequence. The subsets may range in size from 5 nucleotide residues to the entire nucleotide sequence minus one nucleic acid residue. When used in reference to an amino acid sequence refers to subsets of that amino acid sequence. The subsets may range in size from 2 amino acid residues to the entire amino acid sequence minus one amino acid residue.

As used herein, the term “antisense” is used in reference to DNA or RNA sequences which are complementary to a specific nucleotide sequence. Antisense may be produced by any method, including synthesis by splicing the gene(s) of interest in a reverse orientation to a viral promoter which permits the synthesis of a coding strand. Once introduced into a cell, a transcribed strand may combine with natural nucleic acids produced by the cell to form duplexes. These duplexes can then block either the further transcription or translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

As used herein, the terms “polyadenylation”, “polyadenylation of a gene” are similar and interchangeable. Generally, polyadenylation denotes the process and means of adding a polyadenylic acid (poly(A)) tail, i.e., multiple adenosine monophosphates, to an RNA molecule. In particular, polyadenylation may denote the process and means of adding a poly(A) tail to a pre-mRNA molecule, in the process of producing mature mRNA. The reference to polyadenylation particularly aims at native polyadenylation such as occurs under normal physiological conditions. Sequence elements required for polyadenylation refer particularly to cis elements in the sequence of pre-mRNA which the cellular polyadenylation machinery recognises such as to which it binds, such as for example the polyadenylation signal. These sequence such as the polyadenylation signal may vary between groups of eukaryotes. For example, in humans the polyadenylation signal sequence may typically be AATAAA (i.e., AAUAAA in RNA such as pre-mRNA), but variants of it exist, such as ATTAAA.

As used herein, the term “siRNA” refers to either small interfering RNA, short interfering RNA, or silencing RNA. Generally, siRNA comprises a class of double-stranded RNA molecules, approximately 15-25 nucleotides in length comprising a sense strand and an antisense strand. siRNA can be involved in RNA interference (RNAi) pathways and/or RNAi-related pathways. wherein the compounds interfere with either gene expression or mRNA translation.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

As used herein, the term “hybridize”, “hybridizing” or “hybridization” is used in reference to the pairing of complementary nucleic acids using any process by which a strand of a particular nucleic acid joins with a complementary strand of a target nucleotide sequence through base pairing to form a hybridization complex. A particular nucleic acid (e.g., an siRNA) may hybridize to either a portion of a target nucleotide sequence or the full length of the target nucleotide sequence (e.g., an mRNA). Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents exemplary data showing that DUX4-targeted siRNAs reduce DUX4 luciferase activity. The screen assays the efficacy of knockdown with siRNAs targeting different regions of the DUX4 coding sequence.

FIGS. 2 A&B present exemplary data showing that DUX4-targeting siRNAs reduce DUX4 target gene expression in primary FSHD patient myoblasts.
FIG. 2A: Schematic showing the “pre-differentiation” treatment paradigm in primary FSHD myoblasts.
FIG. 2B: qPCR assays of DUX4 target gene and muscle differentiation marker expression following “pre-differentiation” treatment with the indicated siRNAs at 2 μM. Each dot corresponds to an individual culture and is shown as the mean±SEM.

FIG. 3A-D present exemplary data showing that DUX4-targeting siRNAs DU01 and DU05 reduce DUX4 target gene expression in primary FSHD patient myoblasts in a dose dependent manner.
FIG. 3A: Schematic showing the “pre-differentiation” treatment paradigm in primary FSHD myoblasts
FIG. 3B: qPCR assays of DUX4 target gene and muscle differentiation marker expression after DU01 or DU05 treatment at the indicated concentration treated using the “pre-differentiation” treatment paradigm. Data is shown as fold-change normalized to the non-targeting condition (NTC).
FIG. 3C: Schematic showing the “post-differentiation” treatment paradigm in primary FSHD myoblasts
FIG. 3D: qPCR assays of DUX4 target gene and muscle differentiation marker expression after DU01 or DU05 treatment at the indicated concentration treated using the “post-differentiation” treatment paradigms. Data is shown as fold-change normalized to the NTC condition

FIG. 4A-C present exemplary data showing docosanoic acid (DCA) lipid conjugation enhanced oligonucleotide delivery to muscle.
FIG. 4A: A heatmap display of a screen of lipid conjugates and their accumulation in different tissues after systemic delivery.
FIG. 4B: Top Panel: Quantification of thigh diameter from the indicated treatment groups. Each dot corresponds to measurements from one mouse. Bottom Panel: Representative images of muscles in the thigh region for each treatment group. The outline color corresponds to same color in the graph above. Biscans et al., (2021).
FIG. 4C: qPCR assays quantifying Mstn expression in the gastrocnemius, quadriceps and heart muscles shown as a percent of the NTC condition. Biscans et al., (2021).

FIGS. 5 A&B present exemplary data showing siRNA-mediated decreases DUX4 and DUX4 target gene expression in FSHD xenograft muscle from three (3) different patients (17A, 12A or 114A). Data is shown as fold-change normalized to the average of the NTC condition. Each dot represents the average of the two xenografts in each mouse and data is shown as the mean±SEM for each condition.
FIG. 5A: Schematic of muscle xenograft and siRNA treatment protocol.
FIG. 5B: qPCR assays quantifying DUX4 or DUX4 target gene or muscle differentiation gene expression in DCA-NTC or DCA-DU01 treated mice after 2 20 mg/kg subcutaneous injections two (2) days apart.

FIG. 6 presents an exemplary DUX4 messenger ribonucleic acid sequence (SEQ ID NO: 17). Annotations show exemplary siRNA target sequences for DU01 (shaded), DU04 (underlined), DU05 (bold) and DU07 (italics).

FIGS. 7A&B present exemplary data showing screening of the effect of various DUX4 antisense compounds on DUX4 target gene signature and muscle differentiation biomarker expression.

FIG. 7A: Effects of DU01, DU04, DU05, DU07 and SEQ ID NO: 65 of Belayew et al. (2021) on DUX4 target gene signature mRNA expression from TRIM43, LEUTX and MBD3L2.

FIG. 7B: Effects of DU01, DU04, DU05, DU07 and SEQ ID NO: 65 of Belayew et al. (2021) on muscle differentiation as measured by the biomarkers of MYH8, MYH1 and CKM.

### DETAILED DESCRIPTION OF THE INVENTION

The present invention is related to the treatment of muscular dystrophy, specifically FSHD1 and FSHD2. In particular, compositions of short interfering ribonucleic acids are contemplated that are targeted to different regions of a DUX4 messenger riboucleic acid molecule. When applied as a therapeutic method, the short interfering ribonucleic acids reduce DUX4 protein translation to mediate a therapeutic response in human facioscapulohumeral muscular dystrophy myotubes ex vivo and FSHD muscle xenografts in vivo in mice.

### I. Muscular Dystrophy

### A. Overview

Muscular dystrophy (MD) has been reported to be a group of muscle diseases that results in increasing weakening and breakdown of skeletal muscles over time. The disorders differ in which muscles are primarily affected, the degree of weakness, how fast they worsen, and when symptoms begin. Many patients will eventually become unable to walk and carry out motor functions such as raising arms and smiling, while some FSHD patients have associated problems in other organs such as hearing and vision. Muscular dystrophy has been linked to as many as thirty (30) different genetic disorders that are usually classified into categories. The most common type is Duchenne muscular dystrophy (DMD), which typically affects males beginning around the age of four. Other types of common dystrophies include Becker muscular dystrophy, facioscapulohumeral muscular dystrophy (FSHD), limb-girdle muscular dystrophy, and myotonic dystrophies. These disorders are believed to be caused by mutations in genes that are involved in the expression and functions of muscle proteins or in the case of FSHD, the abnormal expression of the DUX4 mRNA and protein causing muscle toxicity. These mutations can either be inherited or de novo and may be X-linked recessive, autosomal recessive, or autosomal dominant. “NINDS Muscular Dystrophy Information Page” National Institute of Neurological Disorders and Stroke, ninds.nih.gov/disorders/md/md (2016); and “Muscular Dystrophy: Hope Through Research”. National Institute of Neurological Disorders and Stroke, ninds.nih.gov/disorders/md/detail md (2016). See, Table 1.

TABLE 1
Exemplary Types Of Muscular Dystrophy

| | | Encoded | |
|---|---|---|---|
| Disorder name | Genetic Mutations | Protein | Description |
| Becker muscular | DMD | Dystrophin | Becker muscular |
| dystrophy | X chromosome | A protein that is | dystrophy (BMD) |
| | deletion | part of a | is a less severe |
| | duplication missense | complex | variant of |
| | | structure | Duchenne muscular |
| | | involving | dystrophy and is |
| | | several other | caused by the |
| | | protein | production of a |
| | | components. The | truncated, but |
| | | “dystrophin- | partially functional |
| | | glycoprotein | form of dystrophin. |
| | | complex” helps | Survival is usually |
| | | anchor the | into old age and |
| | | structural | usually affects only |
| | | skeleton | boys. |
| | | (cytoskeleton) | |
| | | within the | |
| | | muscle cells, | |
| | | through the | |
| | | sarcolemma of | |
| | | each cell, to the | |
| | | extracellular | |
| | | matrix. | |
| Congenital muscular | COL6A | Collagen VI | General muscle |
| dystrophy | nonsense p.Gln889* | A component of | weakness and |
| | p.Pro260_Lys261insProPro | the extracellular | possible joint |
| | small insertion. | matrix which | deformities, disease |
| | | forms a | progresses slowly, |
| | | microfibrillar | and lifespan is |
| | | network that is | shortened. Muscle |
| | | found in close | degeneration may |
| | | association with | be mild or severe. |
| | | the cell and | May show severe |
| | | surrounding | brain |
| | | basement | malformations, |
| | | membrane. | such as |
| | | Disorders result | lissencephaly and |
| | | in a combined | hydrocephalus. |
| | | muscle and | |
| | | connective tissue | |
| | | involvement, | |
| | | including | |
| | | weakness, joint | |
| | | laxity and | |
| | | contractures, and | |
| | | abnormal skin | |
| | | findings. | |
| Duchenne muscular | DMD | Dystrophin | Generally affects |
| dystrophy | X chromosome | A protein that is | only diagnosed by |
| | nonsense frameshift | part of a | age-related |
| | | complex | difficulty in |
| | | structure | walking. Lifespans |
| | | involving | range from 15 to |
| | | several other | 45. Due to defects |
| | | protein | in this assembly, |
| | | components. The | contraction of the |
| | | “dystrophin- | muscle leads to |
| | | glycoprotein | disruption of the |
| | | complex” helps | outer membrane of |
| | | anchor the | the muscle cells |
| | | structural | and eventual |
| | | skeleton | weakening and |
| | | (cytoskeleton) | wasting of the |
| | | within the | muscle. |
| | | muscle cells, | |
| | | through the | |
| | | sarcolemma of | |
| | | each cell, to the | |
| | | extracellular | |
| | | matrix. | |
| Distal muscular | DYSF | Dysferlin | Age at onset is |
| dystrophy | human chromosome 2p13 | Also known as | about 20 to 60 |
| | Pro791Arg | dystrophy- | years; symptoms |
| | C1939G | associated fer-1- | include weakness |
| | G3370T 3746delG | like protein. | and wasting of |
| | 4870delT | Anoctamin 5 | muscles of the |
| | ANO5 | An intracellular | hands, forearms, |
| | | calcium- | and lower legs; |
| | | activated | progress is slow |
| | | chloride channel. | and not life- |
| | | | threatening. |
| | | Defective | Miyoshi myopathy |
| | | plasma | causes initial |
| | | membrane repair | weakness in the |
| | | by loss of | calf muscles. |
| | | annexin | |
| | | trafficking. | |
| Emery-Dreifuss | EMD | Emerin | Present in |
| muscular dystrophy | X-linked recessive | Emerin, together | childhood and the |
| | autosomal dominant | with LEMD3, is | early teenaged |
| | autosomal recessive | a LEM domain- | years with |
| | p.Asp6Glyfs*27 | containing | contractures. |
| | | integral protein | Clinical signs |
| | | of the inner | include muscle |
| | | nuclear | weakness and |
| | | membrane in | wasting, stating in |
| | | vertebrates. | the distal limb |
| | | Emerin is highly | muscles and |
| | | expressed in | progressing to |
| | | cardiac and | involve the limb- |
| | | skeletal muscle. | girdle muscles. |
| | | In cardiac | Most patients also |
| | | muscle, emerin | suffer from cardiac |
| | | localizes to | conduction defects |
| | | adherens | and arrhythmias |
| | | junctions. | and dilated |
| | LMNA | Lamin A/C | cardiomyopathy. |
| | R377H | Belongs to the | |
| | | family of | |
| | | laminin nuclear | |
| | | proteins. | |
| Facioscapulohumeral | DUX4 misexpression | Double Homeobox 4 | Progressive |
| muscular dystrophy | autosomal dominant when | A transcriptional | weakness, initially |
| | associated with the collapse | activator of | in the muscles of |
| | of the D4Z4 repeats to 10 or | paired-like | the face, shoulders, |
| | less repeats containing | homeodomain | and upper arms. |
| | DUX4 disease gene as the | transcription | Additional muscles |
| | terminal D4Z4 repeat on | factor 1 | are often affected. |
| | chromosome 4 (FSHD 1). | (PITX1). | Symptoms usually |
| | Recessive when associated | | manifest in |
| | with associated with | | adolescence, but |
| | mutations in SMCHD1 or | | clinical disease can |
| | DNMT3B (FSHD 2) | | also manifest in |
| | Both FSHD1 and FSHD2 | | early childhood or |
| | require a specific permissive | | adults. Affected |
| | haplotype (pathogenic | | individuals can |
| | variant) of chromosome 4 | | become severely |
| | (4qA; 4qA-L) that contains a | | disabled, with 20% |
| | polymorphism that creates a | | requiring a wheel |
| | polyadenylation signal | | chair by age 50. |
| | (ATTAAA) distal to the | | Penetrance and |
| | D4Z4 array | | severity seem to be |
| | 30% de novo mutations | | lower in females |
| | | | compared to males. |
| | | | The cause is |
| | | | epigenetic de- |
| | | | repression of |
| | | | DUX4, which |
| | | | requires two |
| | | | different types |
| | | | mutations/genetic |
| | | | variants: one |
| | | | mutation causing |
| | | | demethylation of |
| | | | the DUX4 region |
| | | | either through |
| | | | collapse of the |
| | | | D4Z4 repeats or |
| | | | loss of function |
| | | | mutations of |
| | | | proteins (SMCHD1 |
| | | | or DNMT3B) |
| | | | required for proper |
| | | | repressive |
| | | | methylation of the |
| | | | locus, allowing |
| | | | DUX4 |
| | | | transcription, |
| | | | associated with |
| | | | another pathologic |
| | | | sequence variant |
| | | | (e.g., 4qA) |
| | | | containing a |
| | | | polyadenylation |
| | | | signal sequence |
| | | | downstream of |
| | | | DUX4, providing |
| | | | stability to DUX4 |
| | | | messenger RNA |
| | | | and increased |
| | | | likelihood of its |
| | | | translation. |
| Limb-girdle | DYSF | Dysferlin | Affects both boys |
| muscular dystrophy | human chromosome 2p13 | Also known as | and girls. Muscle |
| | autosomal recessive | dystrophy- | weakness, affecting |
| | autosomal dominant | associated fer-1- | both upper arms |
| | Pro791Arg | like protein. | and legs. The |
| | ANO5 | Anoctamin 5 | recessive LGMDs |
| | | An intracellular | are more frequent |
| | | calcium- | than the dominant |
| | | activated | forms, and usually |
| | | chloride channel. | have childhood or |
| | | | teenaged onset. The |
| | | | dominant LGMDs |
| | | | usually show adult |
| | | | onset. Some of the |
| | | | recessive forms |
| | | | have been |
| | | | associated with |
| | | | defects in proteins |
| | | | that make up the |
| | | | dystrophin- |
| | | | glycoprotein |
| | | | complex. Though a |
| | | | person normally |
| | | | leads a normal life |
| | | | with some |
| | | | assistance, in some |
| | | | extreme cases, |
| | | | death from LGMD |
| | | | occurs due to |
| | | | cardiopulmonary |
| | | | complications. |
| Myotonic muscular | DMPK | Dystrophia | Presents with |
| dystrophy | autosomal dominant | myotonica | myotonia (delayed |
| | CTG repeat expansion | protein kinase | relaxation of |
| | | (DMPK) | muscles), as well as |
| | | A Ser/Thr | muscle wasting and |
| | | protein kinase | weakness. Varies in |
| | | homologous to | severity and |
| | | the MRCK p21- | manifestations and |
| | | activated kinases | affects many body |
| | | and the Rho | systems in addition |
| | | family of | to skeletal muscles, |
| | | kinases. [8] | including the heart, |
| | ZNF9 (also known as CNBP) | ZNF9/CNBP | endocrine organs, |
| | CCTG repeat expansion in | A protein | and eyes. |
| | intron 1 | containing seven | |
| | | (7) zinc finger | |
| | | domains and is | |
| | | believed to | |
| | | function as an | |
| | | RNA-binding | |
| | | protein. | |
| | | Also known as, | |
| | | cellular nucleic | |
| | | acid-binding | |
| | | protein (CNBP). | |
| Oculopharyngeal | PABPN1 | Polyadenylate- | Age at onset is 40 |
| muscular dystrophy | Encoding poly-A short | binding protein | to 70 years; |
| | tandem repeats for GCN[11] | 2 (PABP-2) | symptoms affect |
| | to GCN[17]] | (also known as | muscles of eyelids, |
| | | polyadenylate- | face, and throat |
| | | binding nuclear | followed by pelvic |
| | | protein | and shoulder |
| | | 1(PABPN1)) | muscle weakness. |
| | | A protein that is | |
| | | a member of the | |
| | | poly(A)-binding | |
| | | protein family | |
| | | that regulate the | |
| | | translation of | |
| | | some genes into | |
| | | functional | |
| | | proteins. | |

The signs and symptoms of muscular dystrophy include, but are not limited to, progressive muscular wasting, poor balance, scoliosis (i.e., curvature of the spine and the back), progressive inability to walk, waddling gait, calf deformation, limited range of movement, respiratory difficulty, cardiomyopathy, muscle spasms and/or gowers' sign.

### B. Facioscapulohumeral Muscular Dystrophy

Facioscapulohumeral muscular dystrophy (FSHD) is a genetic muscular dystrophy that causes weakening of muscles in the face, shoulder girdle and upper body and progresses to include loss of ambulation and severe clinical disability. FSHD prevalence is between 1 in 20,000 and 1 in 8,000 people with about 15,000-30,000 patients residing in the United States. The genetic cause of FSHD stems from a contraction (deletion) in the 4q35 region of chromosome 4 which leads to hypomethylation of the D4Z4 repeat locus, relaxation of chromatin and expression of the transcription factor DUX4 in skeletal muscle. DUX4 expression is toxic to muscle causing myotube muscle cells ex vivo to die within 24 hours. While DUX4 expression is extremely low and hard to quantify, with expression in only 1 in 1000 muscle cells, it promotes the expression of hundreds of downstream target genes that are expressed at higher levels and are used as a proxy for quantifying DUX4 expression.

FSHD is the third most commonly diagnosed type of muscular dystrophy in the United States and has no FDA approved treatment or cure. While it is still considered a rare disease, it is believed that an efficacious therapeutic is needed and can improve patient's lives. Currently, clinical outcomes and endpoint measures for an FSHD clinical trial are not well defined. A hallmark of the disease is the sometimes slow and sporadic progression of muscle weakness and patient to patient variability with affected muscles. Therefore, it remains challenging to design clinical trials and to identify a potentially responsive cohort of FSHD patients for inclusion.

Facioscapulohumeral muscular dystrophy (FSHD) is a type of muscular dystrophy that preferentially weakens the skeletal muscles of the face, those that position the scapula, and those in the upper arm, overlying the humerus bone (humeral). Weakness of the scapular muscles causes an abnormally positioned scapula. Other areas of the body usually develop weakness as well, such as the abdomen and lower leg, causing foot drop. The two sides of the body are often affected unequally. Symptoms typically begin in early childhood and become noticeable in the teenage years, with 95% of affected individuals manifesting disease by age 20 years.

FSHD is caused by complex genetic changes involving the D4Z4 repeat locus near the telomere on Chromosome 4. The FSHD disease gene DUX4, is encoded by terminal D4Z4 repeat sequences. Typically, DUX4 is expressed during the early development in the male germ line, but not postnatally in somatic tissues. In FSHD, DUX4 is inadequately repressed within muscle tissue. For the majority of individuals (e.g., 95% of FSHD cases), aberrant DUX4 expression is the result of a reduction in the number of D4Z4 macrosatellite repeats (e.g, ˜less than 10 repeats) and results in D4Z4 contraction (FSHD type 1: FSHD1). D4Z4 contraction leads to hypomethylation of the contracted D4Z4 locus and derepression of the DUX4 gene in muscle. Another 5% of FSHD cases are associated with mutations in chromatin modifying genes (e.g. SMCHD1 or DNMT3B) that disrupt the hypomethylation of the D4Z4 locus also relieving the epigenetic repression of DUX4 (FSHD type 2: FSHD2).

Regardless of the type of genetic driver mutation, disease can only result if the individual has a DUX4 allele associated with the 4qA haplotype which is a common variant (e.g., approximately 50% of alleles in population) at the 3′ end of the DUX4 locus. The 4qA haplotype encodes a functional polyadenylation signal sequence (PAS) that permits a stable accumulation of aberrantly expressed DUX4 transcripts mis-expressed in FSHD1 and FSHD2 muscle, whereas other non-permissive, common haplotypes, such as 4qB, do not function as PAS sites and are not associated with disease progression. Notably, DUX4 expressed in normal germline development utilizes a distal PAS site that is not 4qA sequence dependent. FSHD1 follows an autosomal dominant inheritance pattern, meaning each child of an affected individual has a 50% chance of also being affected.

DUX4 is a sequence-specific transcription factor that regulates a large number of germ line genes that are mis-expressed in FSHD muscle in patients and in differentiated myotube cell cultures derived from muscles of FSHD patients. Mis-expression of DUX4 in myotubes in vitro and muscle fibers in vivo causes muscle damage through cell death and the production of local inflammation, although the exact mechanism of muscle destruction remains poorly defined.

There is no known cure for FSHD, and no pharmaceuticals have proven effective for altering the disease course. Symptoms can be addressed with physical therapy, bracing, and reconstructive surgery. Surgical fixation of the scapula to the thorax is effective in reducing shoulder symptoms in select cases. FSHD is the third most common genetic disease of skeletal muscle affecting 1 in 8,333 to 1 in 15,000 people. Prognosis is extremely variable, with some being severely disabled by age 10 and others never facing significant limitations, although up to 20% of all affected individuals require use of a wheelchair or mobility scooter during adolescence. Life expectancy is highly variable, dependent on disease onset, quality of care and respiratory sufficiency.

Because a 4qA allele comprising a functional PAS facilitates disease progression of FSHD, genome editing approaches that disrupt this sequence can be expected to treat this disorder. However, as of the present invention, genome editing has been unsuccessful to disrupt the PAS site and prevent DUX4 expression in myotubes from FSHD patients. Jones et al., “Silencing Of DUX4 By Recombinant Gene Editing Complexes” US2020/0017842; and Das et al., “Genome Editing of the Disease Locus D4Z4 as a Means to Ameliorate Gene Misregulation in Facioscapulohumeral Muscular Dystrophy” Thesis, DigiNole:FSU's Digital Repository, fsu.digital.flvc.org/islandora/object/fsu %3A657913, Florida State University. (2018). These studies indicate that deletion of the PAS sequence by Cas9 editing can reduce DUX4 expression. However, a recent study of PAS modification using TALENs suggests that elimination of the PAS sequence may not prevent DUX4 expression. Joubert et al., “Gene Editing Targeting the DUX4 Polyadenylation Signal: A Therapy for FSHD?” J. Personalized. Med. 11(1):7 (2021); dx.doi.org/10.3390/jpm11010007. Thus, it is unclear whether disruption of the PAS sequence by nucleases or base editors or prime editors can reduce DUX4 expression to therapeutic levels.

### II. Conventional DUX4 Mediated Muscular Dystrophy Therapeutics

The relationships between the DUX4 gene and muscular dystrophy is complex and has been recently reviewed. Banerji et al., “Pathomechanisms and biomarkers in facioscapulohumeral muscular dystrophy: roles of DUX4 and PAX7” EMBO Mol Med (Article e13695 (2021).

DUX4 can inhibit the functions of myogenic regulatory factors MYOD and MYOGENIN that are required for myogenic differentiation and disrupt the enhancer of MYF5. Bosnakovski et al (2008) “An isogenetic myoblast expression screen identifies DUX4-mediated FSHD-associated molecular pathologies” EMBO J 27: 2766-2779; and Bosnakovski et al. (2018) “Low level DUX4 expression disrupts myogenesis through deregulation of myogenic gene expression” Sci Rep 8:16957. DUX4 may also indirectly activate HEY1, a myogenic repressor. Young et al, “DUX4 binding to retroelements creates promoters that are active in FSHD muscle and testis” PLoS Genet 9:e1003947 (2013). Apoptosis is induced by DUX4 in many cell types and species. Kowaljow et al, “The DUX4 gene at the FSHD1A locus encodes a pro-apoptotic protein” Neuromuscul Disord 17:611-623 (2007); and DeSimone et al, “Cellular and animal models for facioscapulohumeral muscular dystrophy” Dis Models Mech 13:dmm046904 (2020). While DUX4 induces p53-dependent apoptosis, it drives apoptosis in TP53-null mice too, possibly via upregulation of p21. Wallace et al, “DUX4, a candidate gene for facioscapulohumeral muscular dystrophy, causes p53-dependent myopathy in vivo” Ann Neurol 69:540-552 (2010); and Bosnakovski et al, “p53-independent DUX4 pathology in cell and animal models of facioscapulohumeral muscular dystrophy” Dis Models Mech 10:1211-1216 (2017), respectively. DUX4 may also force apoptosis by affecting mitochondrial function and sensitizing cells to oxidative stress via disruption of: i) the glutathione redox pathway (Bosnakovski et al, “An isogenetic myoblast expression screen identifies DUX4-mediated FSHD-associated molecular pathologies” EMBO J 27:2766-2779 (2008)); ii) induction of reactive oxygen species (ROS) (Dmitriev et al, “Cancer-related genes in the transcription signature of facioscapulohumeral dystrophy myoblasts and myotubes” J Cell Mol Med 18:208-217 (2016)); iii) HIF1 α signaling ((Banerji et al, “beta-Catenin is central to DUX4-driven network rewiring in facioscapulohumeral muscular dystrophy” JR Soc Interface 12:20140797 (2015); Banerji et al, “PAX7 target genes are globally repressed in facioscapulohumeral muscular dystrophy skeletal muscle” Nat Commun 8:2152 (2017); and Lek et al, “Applying genome-wide CRISPR-Cas9 screens for therapeutic discovery in facioscapulohumeral muscular dystrophy” Sci Transl Med 12:eaay0271 (2020); and iv) increased c-MYC and stabilization of dsRNA (Shadle et al, “DUX4-induced dsRNA and MYC mRNA stabilization activate apoptotic pathways in human cell models of facioscapulohumeral dystrophy” PLoS Genet 13:e1006658 (2017)).

DUX4 target genes have been reported to be significantly elevated in FSHD muscle biopsy studies profiled by RNA-seq. Yao et al, “DUX4-induced gene expression is the major molecular signature in FSHD skeletal muscle” Hum Mol Genet 23:5342-5352 (2014); Wang et al, “MRI-informed muscle biopsies correlate MRI with pathology and DUX4 target gene expression in FSHD” Hum Mol Genet 28:476-486 (2019); Wong et al, “Longitudinal measures of RNA expression and disease activity in FSHD muscle biopsies” Hum Mol Genet 29:1030-1043 (2020). In vivo models of FSHD are necessary to both understand pathomechanisms and test potential therapeutics. Muscle-specific DUX4 expression in mouse can model muscle pathology, including inflammatory changes. However, DUX4 expression dynamics or levels are not accurate without endogenous human regulatory mechanisms, although this is difficult to achieve. Furthermore, there are limitations in overlap between DUX4 target genes in mouse and humans. Krom et al, “Intrinsic epigenetic regulation of the D4Z4 macrosatellite repeat in a transgenic mouse model for FSHD” PLoS Genet 9:e1003415 (2013); and Knopp et al, “DUX4 induces a transcriptome more characteristic of a less-differentiated cell state and inhibits myogenesis” J Cell Sci 129:3816-3831 (2016). Xenograft models that graft FSHD muscle or cell culture might be more representative. Zhang et al, “Human skeletal muscle xenograft as a new preclinical model for muscle disorders” Hum Mol Genet 23:3180-3188 (2014); Moyle et al, “Ret function in muscle stem cells points to tyrosine kinase inhibitor therapy for facioscapulohumeral muscular dystrophy” eLife 5:e11405 (2016); and Mueller et al, “Muscle xenografts reproduce key molecular features of facioscapulohumeral muscular dystrophy” Exp Neurol 320:113011 (2019).

III. DUX4-mRNA Targeted siRNAs

In one embodiment, the present invention contemplates a composition comprising or consisting of a DUX4-mRNA targeted short interfering ribonucleic acid (siRNA). In one embodiment, the DUX4-mRNA targeted siRNA is double stranded and comprises a sense strand and an antisense strand. In one embodiment, the sense strand is fifteen (15) nucleotides. In one embodiment, the DUX4-mRNA targeted siRNA molecule comprises or consists of SEQ ID NO: 1 (DU01). In one embodiment, the DUX4-mRNA targeted siRNA molecule comprises or consists of SEQ ID NO: 3 (DU04). In one embodiment, the DUX4-mRNA targeted siRNA molecule comprises or consists of SEQ ID NO: 5 (DU05). In one embodiment, the DUX4-mRNA targeted siRNA molecule comprises or consists of SEQ ID NO: 7 (DU07). In one embodiment, the DUX4-mRNA targeted siRNAs are complementary to at least a portion of a DUX4 mRNA target sequence. See, Tables 2-5.

TABLE 2
Representative DUX4 Double Stranded siRNA Molecule Sequences

| | | |
|---|---|---|
| siRNA | Encoded DNA | mRNA |

siRNA Sense Strand Sequences

| | | |
|---|---|---|
| DU01 | TTACATCTCCTGGAT | UUACAUCUCCUGGAU |
| DU04 | GGATTAGAGTTACAT | GGAUUAGAGUUACAU |
| DU05 | AGAGTTACATCTCCT | AGAGUUACAUCUCCU |
| DU07 | CTGGATTAGAGTTAC | UGGAUUAGAGUUAC |

siRNA Antisense Strand Sequence

| | | |
|---|---|---|
| DU01 | TTTAATATATCTCTGAACTA | UUUAAUAUAUCUCUGAACUA |
| DU04 | TCTCTGAACTAATCATCCAG | UCUCUGAACUAAUCAUCCAG |
| DU05 | ATATATCTCTGAACTAATCA | AUAUAUCUCUGAACUAAUCA |
| DU07 | TCTGAACTAATCATCCAGGA | UCUGAACUAAUCAUCCAGGA |

TABLE 3
Representative In Vitro DUX4 Double Stranded siRNA Molecule Formulations

| | |
|---|---|
| siRNA | Formulation Code |

Sense strand

| | |
|---|---|
| DU01 | fC.mA.fG.mA.fG.mA.fU.mA.fU.mA.fU.mU.fA#mA#fA1 |
| DU04 | fU.mG.fA.mU.fU.mA.fG.mU.fU.mC.fA.mG.fA#mG#fA1 |
| DU05 | fA.mG.fU.mU.fC.mA.fG.mA.fG.mA.fU.mA.fU#mA#fA1 |
| DU07 | fG.mA.fU.mG.fA.mU.fU.mA.fG.mU.fU.mC.fA#mG#fA1 |

Antisense Strand

| | |
|---|---|
| DU01 | PmU.fU.mU.fA.mA.fU.mA.fU.mA.fU.mC.fU.mC.fU#mG#fA#mA#fC#mU#fA |
| DU04 | PmU.fC.mU.fC.mU.fG.mA.fA.mC.fU.mA.fA.mU.fC#mA#fU#mC#fC#mA#fG |
| DU05 | PmU.fU.mA.fU.mA.fU.mC.fU.mC.fU.mG.fA.mA.fC#mU#fA#mA#fU#mC#fA |
| DU07 | PmU.fC.mU.fG.mA.fA.mC.fU.mA.fA.mU.fC.mA.fU#mC#fC#mA#fG#mG#fA |

TABLE 4
Representative In Vivo DUX4 Double Stranded siRNA Molecule Formulations

| | |
|---|---|
| siRNA | Formulation Code |

Sense Strand

| | |
|---|---|
| DU01 | (mU)#(mC)#(mA)(fG)(mA)(fG)(mA)(fU)(mA)(fU)(mA)(mU)(mU)(fA)#(mA)#(mA)- |
| | DCAv1 |

Antisense Strand

| | |
|---|---|
| DU01 | V(mU)#(fU)#(mU)(fA)(fA)(fU)(mA)(fU)(mA)(fU)(mC)(fU)(mC)(fU)#(mG)#(fA)# |
| | (mA)#(mC)#(mU)#(fA)#(mA) |

TABLE 5
Representative DUX4 mRNA Target Sequences

| siRNA | 20-mer | 30-mer |
|---|---|---|
| DU01 | UAGUUCAGAGAUAUAUUAAA | AUGAUUAGUUCAGAGAUAUAUUAAAAUGCC |
| DU04 | CUGGAUGAUUAGUUCAGAGA | AUCUCCUGGAUGAUUAGUUCAGAGAUAUAU |
| DU05 | UGAUUAGUUCAGAGAUAUAU | CUGGAUGAUUAGUUCAGAGAUAUAUUAAAA |
| DU07 | UCCUGGAUGAUUAGUUCAGA | ACAUCUCCUGGAUGAUUAGUUCAGAGAUAU |

Although it is not necessary to understand the mechanism of an invention it is believed that double stranded DUX4 mRNA targeted siRNAs hybridize with at least a portion of the DUX4 mRNA thereby causing destabilization and/or degradation of the DUX4 mRNA. This destabilization and/or degradation concomitantly and potently decreases DUX4 protein translation cells and thereby reduces DUX4 protein levels. In one embodiment, the cell is a human muscular dystrophy cell such as a FSHD cell. As shown below, DU01 DUX4 siRNA significantly decreases DUX4 mRNA levels in vivo using human FSHD patient muscle xenografts. It is further believed that a 50% decrease in DUX4 protein expression provides a therapeutic improvement in FSHD patients.

The data disclosed herein show that DUX4-mRNA targeted siRNAs (e.g., DU01 and DU05) potently decrease DUX4 mRNA levels. In particular, the data further show that not only do these fifteen (15) nucleotide siRNA sense strands result in a reduction in overall DUX4 RNA levels but also reduce mRNA levels of genes that are modulated by DUX4 protein (e.g., DUX4 target gene signatures). These global DUX4-related mRNA level reductions were observed both in vitro using primary FSHD patient derived muscle stem cells and in vivo using a human muscle stem cell xenoengraftment model.

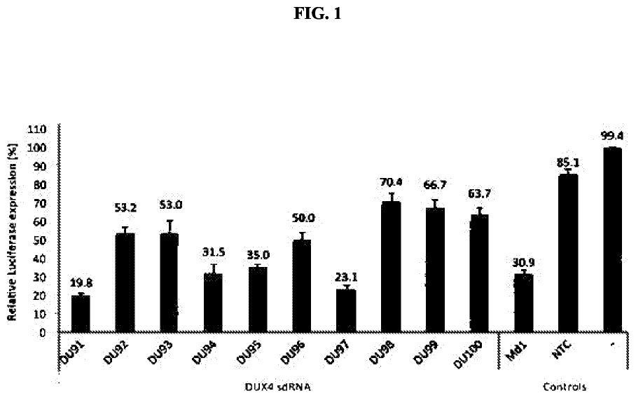

A series of double stranded DUX4-mRNA targeted siRNAs were used, each hybridizing to a different portion of a DUX4 mRNA molecule. See, FIG. 6. A primary screen measured DUX4 luciferase activity in cells treated with siRNAs targeting different coding regions of DUX4 mRNA. Luciferase levels were decreased upon the delivery of the siRNAs DU91 (DU01), DU94 (DU04), DU95 (DU05) and DU97 (DU07). See, FIG. 1.

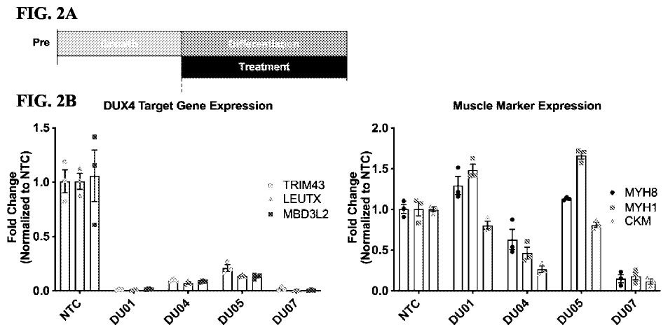

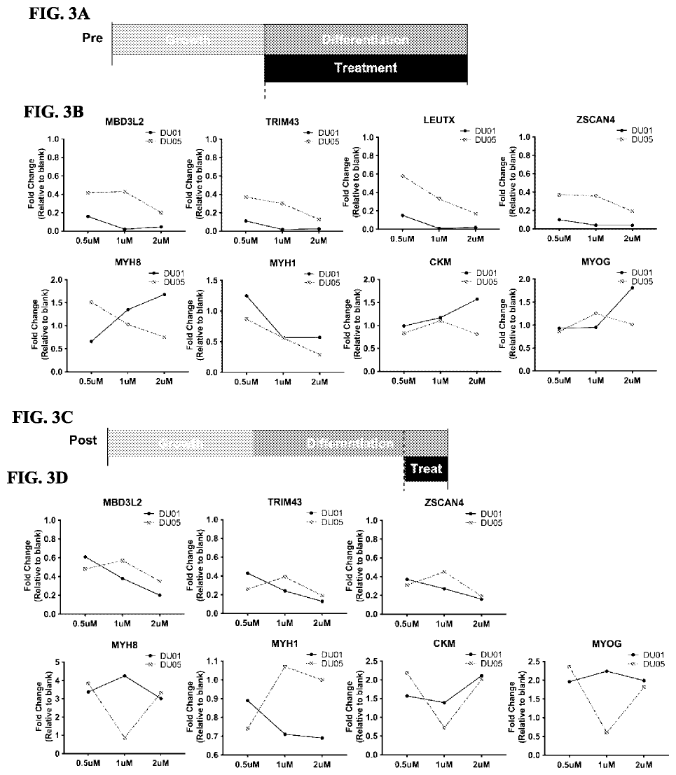

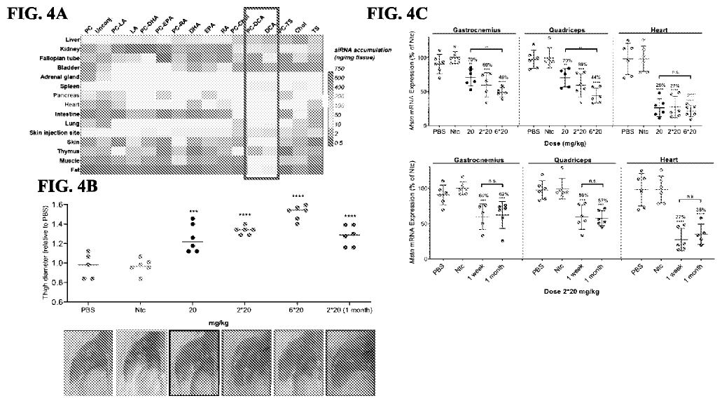

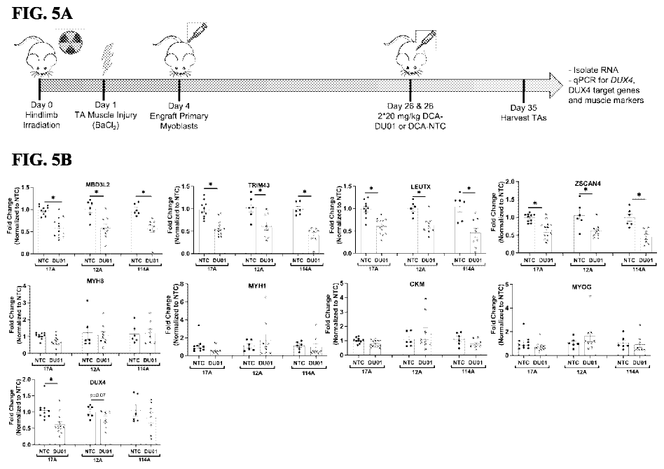

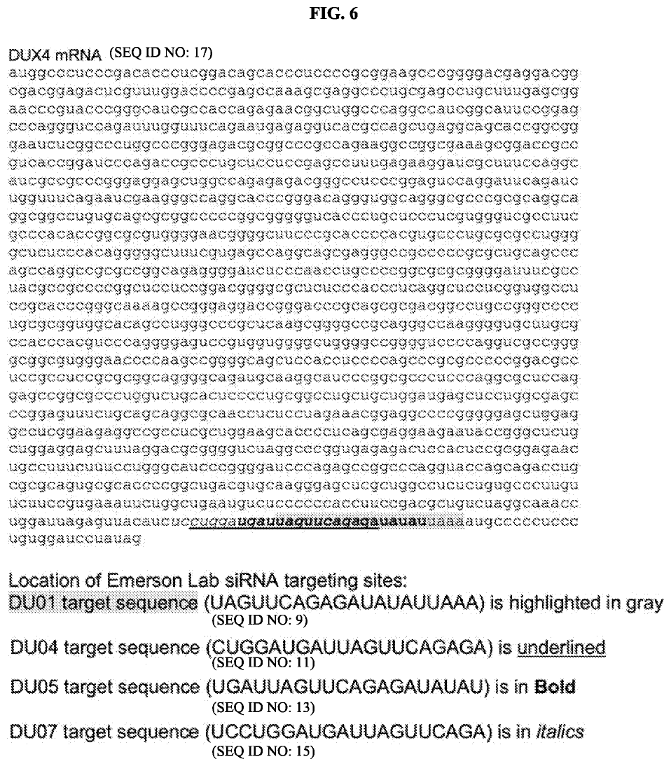

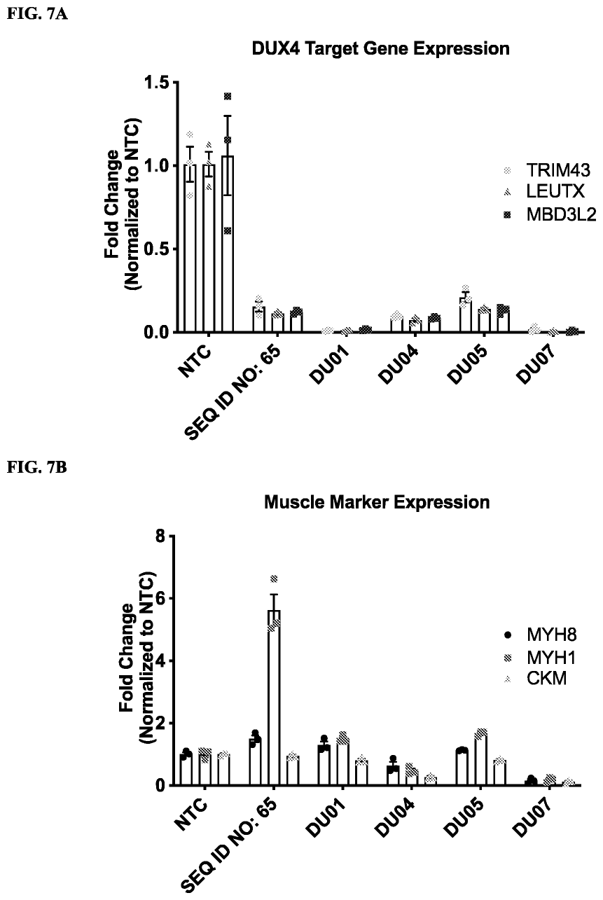

DUX4 expression in cells is extremely low and hard to quantify. In fact, DUX4 protein expression is believed to occur in only 1 in 1000 muscle cells. However, the DUX4 protein promotes the expression of hundreds of downstream target genes, wherein these DUX4 target genes are expressed at higher levels than DUX4 gene expression itself and are generally known to be used as a proxy for quantifying DUX4 gene expression. Consequently, four (4) DUX4 mRNA targeting siRNAs as contemplated herein were assayed for their efficiency to knockdown the expression of three (3) DUX4 target genes (e.g., TRIM43, MBD3L2 and LEUTX) in primary FSHD patient-derived myoblasts using a pre-differentiation treatment paradigm. See, FIG. 2A. Cells were grown to confluence and the DUX4 mRNA-targeting siRNAs (DU01, 04, 05 and 07) or a non-targeting siRNA (NTC) were added at the initiation of myotube differentiation. After 4 days of myotube differentiation, mRNA was isolated for qPCR analysis for expression of each target gene. The data demonstrates that all tested siRNA compounds significantly decreased mRNA levels of TRIM43, MBD3L2 and LEUTX.

The data show that DU01 and DU07 decreased DUX4 target gene mRNA levels most potently. However, it should be noted that the expression of muscle differentiation genes (MYH8, MYH1 and CKM) when treated with either DU04 or DU07 siRNA were also significantly decreased. See, FIG. 2B. Subsequent dose response experiments in a “pre-differentiation” treatment paradigm and a “post-differentiation” treatment paradigm showed a dose-dependent decrease in DUX4 target gene RNA levels in proportion to increasing amounts of DU01 or DU05. See, FIG. 3B and FIG. 3D, respectively. These data show that, as reflected by the mRNA knockdown of DUX4 target gene expression, suggest that DUX4 mRNA levels would necessarily be reduced as well.

In one embodiment, the present invention contemplates a double stranded DUX4-mRNA targeted siRNA conjugated to a lipid such as cholesterol and/or a fatty acid. Biscans et al., (2021). Although it is not necessary to understand the mechanism of an invention, it is believed that lipid-conjugated siRNA significantly improves systemic administration of oligonucleotides to tissues (other than liver) and is designed to overcome the well known roadblocks of extra-hepatic oligonucleotide administration.

Various siRNA-lipid conjugates were screened for preferential accumulation in muscle. The data suggested docosanoic acid (DCA) as the best candidate lipid. See, FIG. 4A. A DCA-conjugated siRNA was created targeting the mouse myostatin gene (Mstn). It is believed that myostatin inhibition results in increased muscle mass. Wildtype mice were treated with a myostatin-targeting siRNA at a frequency of 1, 2 or 6 injections at 20 mg/kg or NTC and harvested the mice one week or one month post siRNA treatment. The data show that every dosing regime significantly increased the muscle mass in the mice, calculated by measuring thigh diameter of Mstn siRNA treated mice relative to PBS injected mice. See, FIG. 4B. This effect persisted for one month post-treatment. In addition to a visual increase in muscle mass, Mstn mRNA expression in the gastrocnemius, quadriceps and heart muscles of treated mice decreased between 30 and 50% as compared to NTC treated mice. As with the increased muscle mass, this decrease in myostatin mRNA expression also persisted for one month after treatment. See, FIG. 4C.

The DCA-conjugated double stranded DUX4-mRNA targeted siRNA delivery platform was combined with a protocol for the efficient and reproducible xenoengraftment of human muscle stem cells into the tibialis anterior (TA) muscles of hindlimb of irradiated and BaCl₂-injured immune-deficient mice. These xenografts form highly humanized regions of differentiated muscle fibers in the mouse TA and can result in a 20-80% humanization of the muscle. This model is well-accepted as a preclinical drug development model using primary biopsy or iPSC-derived myoblasts from patients with several types of muscular dystrophy to assay efficacy of different therapeutic modalities including repurposed small molecule drugs, CRISPR Cas9 genome editing and RNA therapeutics.

Two lipid-conjugated siRNA delivery platforms (e.g., DCA-DU01 and DCA-NTC) were synthesized and systemically delivered in vivo to FSHD-engrafted mice after two weeks of engraftment. See, FIG. 5A. Muscle stem cells from three (3) FSHD patients (17A, 12A and 114A) were engrafted into mice and subjected to DCA-DU01 or DCA-NTC treatment. Two (2) 20 mg/kg injections were delivered 2 days apart. Subsequently, TA muscles were harvested and RNA was isolated one week post treatment for qPCR analysis. DU01 treated mice showed significantly lower levels of DUX4 target gene RNA levels that were 50-70% reduced as compared to the NTC treated mice and all muscle differentiation gene expression remained unchanged. DUX4 RNA levels were significantly decreased in the 17A patient xenografts and close to significant for the other two patients. See, FIG. 5B. These data show that changes in total DUX4 target gene RNA levels reliably predict changes in DUX4 mRNA levels.

DUX4-related DNA antisense and/or RNA interference (RNAi) compounds have been previously suggested as possible therapeutics for FSHD. Belayew et al. “Agents Useful In Treating Facioscapulohumeral Muscular Dystrophy” United States Patent Application Publication No. 2021/0163941 (herein incorporated by reference in its entirety). This reference discloses antisense compounds that are complementary to either genomic splicing sequence elements or polyadenylation sequence elements within, or physically associated with (e.g., pre-mRNA), the DUX4 gene.

One example of a DUX4 pre-RNA binding antisense compound is Belayew's SEQ ID NO: 65. The data presented herein compares FSHD primary myoblasts that were treated with DU01, DU04, DU05, DU07 siRNAs at 2 μM or Belayew's SEQ ID NO: 65 (morpholino) at 10 μM. Cells were treated at the time of differentiation induction and harvested after 4 days of differentiation for RNA isolation and qPCR.

The data demonstrates that DUX4 target gene signatures, including TRIM43, LEUTX and MBD3L2, showed that mRNA knockdown was 10-fold greater using the presently disclosed DU01 or DU07 siRNA compounds when compared to Belayew's SEQ ID NO: 65. See, FIG. 7A. The results also demonstrated that the muscle marker MYH1 was significantly increased when using Belayew's SEQ ID NO: 65 as compared with the NTC and DU01, DU04, DU05 or DU07. See, FIG. 7B. These data provide evidence that the presently disclosed DUX4-mRNA targeted siRNAs are vastly superior to DUX4 antisense that is directed to DUX4 pre-RNA or the DUX4 gene itself.

Belayew et al. also speculates RNAi compounds that might be constructed to bind to the 3′-UTR region of a DUX4 mRNA. Belayew et al. also speculates that their disclosed SEQ ID NOs: 46 and 49, both consisting of nineteen (19) nucleic acids, might serve as potential target sequences for a variety of undisclosed RNAi compounds having lengths ranging from 16-35 nucleotides in length. The data presented herein demonstrates that specific siRNA binding sites that differ by only a few nucleotides result in significant differences in efficacy. For example, even though the presently disclosed DU01, 04, 05 and 07 all consist of fifteen (15) nucleotides, they each have a different mRNA binding site that differ in location by only a few nucleotides. See, FIG. 6. The data shows that these different binding sites are reflected in differences in observed siRNA efficacy of mRNA knockdown. For example, DU01 and DU07 were much more efficacious than either DU04 or DU05. See, FIG. 7A. As such, Belayew's SEQ ID NO; 46 or 49 have no predictive value to guide one of skill in the art to construct any RNAi with any reasonable expectation of success merely by proposing target sequences without empirically validating the binding sites and relative efficacies of specific RNAi compounds.

### III. Polymeric Delivery Systems

The present invention contemplates several drug delivery systems that provide for roughly uniform distribution, have controllable rates of release. A variety of different media are described below that are useful in creating drug delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

One embodiment of the present invention contemplates a drug delivery system comprising therapeutic agents as described herein.

### Microparticles

One embodiment of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

### Liposomes

One embodiment of the present invention contemplates liposomes capable of attaching and releasing therapeutic agents described herein. Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phospholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In some embodiments, the present invention contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes that provide controlled release of at least one therapeutic agent. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### Microspheres, Microparticles And Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze dried medium comprising at least one therapeutic agent is homogenized in a suitable solvent and sprayed to manufacture microspheres in the range of 20 to 90 m. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of therapeutic agent release. Miller et al., Degradation Rates of Oral Resorbable Implants {Polylactates and Polyglycolates: Rate Modification and Changes in PLA/PGA Copolymer Ratios, J. Biomed. Mater. Res., Vol. II:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a therapeutic agent is added to the biodegradable polymer metal salt solution. The weight ratio of a therapeutic agent to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and therapeutic agent is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a therapeutic agent for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-500 μm and composed of biocompatible and biodegradable polymers. Specific polymer compositions of a microsphere can control the therapeutic agent release rate such that custom-designed microspheres are possible, including effective management of the burst effect. ProMaxx® (Epic Therapeutics, Inc.) is a protein-matrix delivery system. The system is aqueous in nature and is adaptable to standard pharmaceutical delivery models. In particular, ProMaxx® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005% -0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a therapeutic agent is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one embodiment, the present invention contemplates microparticles formed by spray-drying a composition comprising fibrinogen or thrombin with a therapeutic agent. Preferably, these microparticles are soluble and the selected protein (i.e., fibrinogen or thrombin) creates the walls of the microparticles. Consequently, the therapeutic agents are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

1. A composition comprising a double stranded DUX4-mRNA targeted short interfering ribonucleic acid (siRNA) comprising a sense strand consisting of fifteen (15) nucleotides.

2. The composition of claim 1, wherein said double stranded DUX4-mRNA targeted siRNA further comprises an antisense strand consisting of twenty (20) nucleotides.

3. The composition of claim 1, wherein said sense strand is complementary to at least a portion of a DUX4 mRNA target sequence.

4. The composition of claim 1, wherein said double stranded DUX4-mRNA targeted siRNA is conjugated to a lipid.

5. The composition of claim 4, wherein aid lipid is docosanoic acid or cholesterol.

6. The composition of claim 1, wherein said composition is a pharmaceutically acceptable composition.

7. The composition of claim 1, wherein said sense strand consists of the sequence UUACAUCUCCUGGAU (SEQ ID NO: 1).

8. The composition of claim 2, wherein said antisense strand consists of the sequence UUUAAUAUAUCUCUGAACUA (SEQ ID NO: 2).

9. The composition of claim 1, wherein said sense strand consists of the sequence GGAUUAGAGUUACAU (SEQ ID NO:3).

10. The composition of claim 2, wherein said antisense strand consists of the sequence UCUCUGAACUAAUCAUCCAG (SEQ ID NO: 4).

11. The composition of claim 1, wherein said sense strand consists of the sequence AGAGUUACAUCUCCU (SEQ ID NO: 5).

12. The composition of claim 2, wherein said antisense strand consists of the sequence AUAUAUCUCUGAACUAAUCA (SEQ ID NO:6).

13. The composition of claim 1, wherein said sense strand consists of the sequence CUGGAUUAGAGUUAC (SEQ ID NO:7).

14. The composition of claim 2, wherein said antisense strand consists of the sequence UCUGAACUAAUCAUCCAGGA (SEQ ID NO: 8).

15. The composition of claim 7, wherein said SEQ ID NO: 1 is complementary to at least a portion of a UAGUUCAGAGAUAUAUUAAA (SEQ ID NO:9) target sequence or at least of portion of a AUGAUUAGUUCAGAGAUAUAUUAAAAUGCC (SEQ ID NO: 10) target sequence.

16. The composition of claim 9, wherein said SEQ ID NO: 3 is complementary to at least a portion of a CUGGAUGAUUAGUUCAGAGA (SEQ ID NO: 11) target sequence or at least a portion of a AUCUCCUGGAUGAUUAGUUCAGAGAUAUAU (SEQ ID NO:12) target sequence.

17. The composition of claim 11, wherein said SEQ ID NO: 5 is complementary to at least a portion of a UGAUUAGUUCAGAGAUAUAU (SEQ ID NO: 13) target sequence or at least a portion of a CUGGAUGAUUAGUUCAGAGAUAUAUUAAAA (SEQ ID NO: 14) target sequence.

18. The composition of claim 13, wherein said SEQ ID NO: 7 is complementary to at least a portion of a UCCUGGAUGAUUAGUUCAGA (SEQ ID NO: 15) target sequence or at least a portion of a ACAUCUCCUGGAUGAUUAGUUCAGAGAUAU (SEQ ID NO:16) target sequence.

19. A method, comprising:
a) providing;
i) a patient exhibiting at least one symptom of a muscular dystrophy disease and comprising a DUX4 messenger ribonucleic acid (mRNA) target sequence; and
ii) a pharmaceutically acceptable composition comprising a double stranded DUX4-mRNA targeted short interfering ribonucleic acid (siRNA) comprising a sense strand that consists of fifteen (15) nucleotides; and
b) administering said pharmaceutically acceptable composition to said patient such that said at least one symptom of said muscular dystrophy disease is reduced.

20. The method of claim 19, wherein said method further comprises hybridizing said double stranded DUX4-mRNA targeted siRNA to at least a portion of said DUX4 mRNA target sequence.

21.-35. (canceled)


SEQUENCE LISTING (17 sequences)

SEQ ID NO: 1 - RNA, 15 nt, synthetic construct
  misc_feature 1..15: note = Description of sequence: DUX4-mRNA targeted siRNA
  misc_feature 1..15: note = Note: double stranded RNA
  source 1..15: mol_type = other RNA
ttacatctcctggat

SEQ ID NO: 2 - RNA, 20 nt, synthetic construct
  misc_feature 1..20: note = Description of sequence: DUX4-mRNA targeted siRNA
  misc_feature 1..20: note = Note: double stranded RNA
  source 1..20: mol_type = other RNA
tttaatatatctctgaacta

SEQ ID NO: 3 - RNA, 15 nt, synthetic construct
  misc_feature 1..15: note = Description of sequence: DUX4-mRNA targeted siRNA
  misc_feature 1..15: note = Note: double stranded RNA
  source 1..15: mol_type = other RNA
ggattagagttacat

SEQ ID NO: 4 - RNA, 20 nt, synthetic construct
  misc_feature 1..20: note = Description of sequence: DUX4-mRNA targeted siRNA
  misc_feature 1..20: note = Note: double stranded RNA
  source 1..20: mol_type = other RNA
tctctgaactaatcatccag

SEQ ID NO: 5 - RNA, 15 nt, synthetic construct
  misc_feature 1..15: note = Description of sequence: DUX4-mRNA targeted siRNA
  misc_feature 1..15: note = Note: double stranded RNA
  source 1..15: mol_type = other RNA
agagttacatctcct

SEQ ID NO: 6 - RNA, 20 nt, synthetic construct
  misc_feature 1..20: note = Description of sequence: DUX4-mRNA targeted siRNA
  misc_feature 1..20: note = Note: double stranded RNA
  source 1..20: mol_type = other RNA
atatatctctgaactaatca

SEQ ID NO: 7 - RNA, 15 nt, synthetic construct
  misc_feature 1..15: note = Description of sequence: DUX4-mRNA targeted siRNA
  misc_feature 1..15: note = Note: double stranded RNA
  source 1..15: mol_type = other RNA
ctggattagagttac

SEQ ID NO: 8 - RNA, 20 nt, synthetic construct
  misc_feature 1..20: note = Description of sequence: DUX4-mRNA targeted siRNA
  misc_feature 1..20: note = Note: double stranded RNA
  source 1..20: mol_type = other RNA
tctgaactaatcatccagga

SEQ ID NO: 9 - RNA, 20 nt, synthetic construct
  misc_feature 1..20: note = Description of sequence: DUX4-mRNA targeted siRNA
  misc_feature 1..20: note = Note: double stranded RNA
  source 1..20: mol_type = other RNA
tagttcagagatatattaaa

SEQ ID NO: 10 - RNA, 30 nt, synthetic construct
  misc_feature 1..30: note = Description of sequence: DUX4-mRNA targeted siRNA
  misc_feature 1..30: note = Note: double stranded RNA
  source 1..30: mol_type = other RNA
atgattagttcagagatatattaaaatgcc

SEQ ID NO: 11 - RNA, 20 nt, synthetic construct
  misc_feature 1..20: note = Description of sequence: DUX4-mRNA targeted siRNA
  misc_feature 1..20: note = Note: double stranded RNA
  source 1..20: mol_type = other RNA
ctggatgattagttcagaga

SEQ ID NO: 12 - RNA, 30 nt, synthetic construct
  misc_feature 1..30: note = Description of sequence: DUX4-mRNA targeted siRNA
  misc_feature 1..30: note = Note: double stranded RNA
  source 1..30: mol_type = other RNA
atctcctggatgattagttcagagatatat

SEQ ID NO: 13 - RNA, 20 nt, synthetic construct
  misc_feature 1..20: note = Description of sequence: DUX4-mRNA targeted siRNA
  misc_feature 1..20: note = Note: double stranded RNA
  source 1..20: mol_type = other RNA
tgattagttcagagatatat

SEQ ID NO: 14 - RNA, 30 nt, synthetic construct
  misc_feature 1..30: note = Description of sequence: DUX4-mRNA targeted siRNA
  misc_feature 1..30: note = Note: double stranded RNA
  source 1..30: mol_type = other RNA
ctggatgattagttcagagatatattaaaa

SEQ ID NO: 15 - RNA, 20 nt, synthetic construct
  misc_feature 1..20: note = Description of sequence: DUX4-mRNA targeted siRNA
  misc_feature 1..20: note = Note: double stranded RNA
  source 1..20: mol_type = other RNA
tcctggatgattagttcaga

SEQ ID NO: 16 - RNA, 30 nt, synthetic construct
  misc_feature 1..30: note = Description of sequence: DUX4-mRNA targeted siRNA
  misc_feature 1..30: note = Note: double stranded RNA
  source 1..30: mol_type = other RNA
acatctcctggatgattagttcagagatat

SEQ ID NO: 17 - RNA, 2072 nt, synthetic construct
  misc_feature 1..2072: note = Description of sequence:exemplary DUX4 messenger RNA
  source 1..2072: mol_type = other RNA
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggacgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccacacagcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagccgcccaggtgccagcacggagcgcctggcggtcaaaagcatacctctgtctgtctttgcccgcttcctggctagacctgcgtgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag